(19) United States
(12) Patent Application Publication
(10) Pub. No.: US 2022/0370197 A1
(43) Pub. Date: Nov. 24, 2022

(54) BALLOON CATHETER FOR PROLAPSED NASAL VALVE PROCEDURE

(71) Applicant: Acclarent, Inc., Irvine, CA (US)

(72) Inventors: Jetmir Palushi, Irvine, CA (US); Alison D. Wright, Newport Beach, CA (US); Marc Dean, Fort Worth, TX (US)

(21) Appl. No.: 17/730,265

(22) Filed: Apr. 27, 2022

Related U.S. Application Data
(60) Provisional application No. 63/190,839, filed on May 20, 2021.

(51) Int. Cl. A61F 2/18; A61B 17/24; A61B 17/34; A61M 25/10; A61L 27/58

(52) U.S. Cl. CPC A61F 2/186; A61B 17/24; A61B 17/3468; A61M 25/1002; A61L 27/58; A61M 2029/025

(57) ABSTRACT

A method is used to insert an implant within a lateral wall of a nose of a patient. The method includes forming an initial incision with a surface of the lateral wall of the nose. A stylet is advanced within the lateral wall of the nose, thereby creating a pocket within the lateral wall of the nose. The stylet is further advanced until an expanding body of the stylet is at least partially housed within the pocket of the lateral wall. The expanding body of the stylet is enlarged to thereby enlarge the pocket within the lateral wall. The implant is placed within the enlarged pocket of the lateral wall.

### PRIORITY

This application claims priority to U.S. Provisional Pat. App. No. 63/190,839, entitled “Balloon Catheter for Prolapsed Nasal Valve Procedure,” filed May 20, 2021, the disclosure of which is incorporated by reference herein, in its entirety.

### BACKGROUND

Nasal breathing may provide a significant amount of airflow to the lungs. If a patient has a prolapsed nasal valve, the lateral wall of the nose (which may include the lower lateral cartilage of the nose, the upper lateral cartilage of the nose, and the mucosal tissue adjacent to both the upper and lower lateral cartilage of the nose) may collapse medially (inwardly) toward the septum as the patient attempts to breathe in via the nasal passageway. This collapse of the lateral wall of the nose may lead to an undesirable decrease in airflow to the lungs via the nasal passageway, as well as other quality of life consequences. Therefore, in some instances, it may be desirable to insert an implant within the interior of the lateral wall of the nose (e.g., the portion of the lateral wall not exposed medially toward the nasal cavity or laterally toward the external environment) for structural support. Structural support created by an implant within the interior of the lateral wall of the nose may inhibit future nasal valve prolapses generated via nasal breathing.

While instruments and methods for performing prolapsed nasal valve procedures are known, it is believed that no one prior to the inventors has made or used the invention described in the appended claims.

### BRIEF DESCRIPTION OF THE DRAWINGS

The drawings and detailed description that follow are intended to be merely illustrative and are not intended to limit the scope of the invention as contemplated by the inventors.

FIG. 1 depicts an exploded perspective view of an instrument that may be used to form an opening within an interior of the lateral nasal wall and insert an implant within the newly formed opening;

FIG. 2 depicts a perspective view of a dissecting balloon catheter of the instrument of FIG. 1;

FIG. 3 depicts a cross-sectional view of the dissecting balloon catheter of FIG. 2, taken along line 3-3 of FIG. 2;

FIG. 4A depicts a perspective view of a distal end of the dissecting balloon catheter of FIG. 2, where an expanding body of the dissecting balloon catheter is in a deflated configuration;

FIG. 4B depicts a perspective view of the distal end of the dissecting balloon catheter of FIG. 2, where the expanding body of FIG. 4A is in an inflated configuration;

FIG. 5A depicts a superior view of the underside of a nose of a patient, with a pair of elevators providing access to a nasal cavity of the patient;

FIG. 5B depicts a superior view of the underside of the nose of the patient, with a pair of elevators providing access to the nasal cavity of the patient; where the instrument of FIG. 1 has been used to access the interior of the lateral wall of the nose and insert an implant within the accessed interior of the lateral wall;

FIG. 6A depicts a perspective view of a distal tip of a guide catheter assembly of the instrument of FIG. 1 being inserted within a nasal cavity of a patient, where the distal tip is adjacent to the lateral wall of the nose defining a portion of the nasal cavity;

FIG. 6B depicts a perspective view of the distal tip of FIG. 6A adjacent to the lateral wall of the nose within the nasal cavity, where a penetrating distal tip of the dissecting balloon catheter is advanced distally past the distal tip to form an initial incision within the lateral wall;

FIG. 6C depicts a perspective view of the distal tip of FIG. 6A adjacent to the lateral wall of the nose within the nasal cavity, where the penetrating distal tip of FIG. 6B is further advanced in a superior direction within the lateral wall to form an initial elevated pocket, where the expanding body of FIG. 4A is in the deflated configuration;

FIG. 6D depicts a perspective view of the distal tip of FIG. 6A adjacent to the lateral wall of the nose within the nasal cavity, where the expanding body of FIG. 4A is in the inflated configuration, thereby expanding the elevated pocket formed in FIG. 6C and the initial incision formed in FIG. 6B;

FIG. 6E depicts a perspective view of the distal tip of FIG. 6A adjacent to the lateral wall of the nose within the nasal cavity, where the expanding body of FIG. 4A is in the deflated configuration, thereby leaving the recently expanded elevated pocket and initial incision formed in FIG. 6D;

FIG. 6F depicts a perspective view of the distal tip of FIG. 6A adjacent to the lateral wall of the nose within the nasal cavity, where the dissecting balloon catheter is retracted inferiorly out of elevated pocket formed in FIG. 6D and proximally into the confines of the guide catheter assembly of FIG. 6A;

FIG. 6G depicts a perspective view of the distal tip of FIG. 6A adjacent to the lateral wall of the nose within the nasal cavity, where the implant of FIG. 1 is advanced with a deployment mechanism into the elevated pocket formed in FIG. 6D via the initial incision formed in FIG. 6B;

FIG. 6H depicts a perspective view of the distal tip of FIG. 6A adjacent to the lateral wall of the nose within the nasal cavity, where the deployment mechanism of FIG. 6G is retracted inferiorly out of the elevated pocket formed in FIG. 6D and into the confines of the guide catheter assembly of FIG. 6A, where the polymer plate implant of FIG. 1 is left within the elevated pocket formed in FIG. 6D.

FIG. 6I depicts a perspective view of the distal tip of FIG. 6A advanced to abut against the incision formed in FIG. 6B such that the distal tip transmits RF energy to seal the incision formed on the lateral wall of the nose within the nasal cavity;

FIG. 7 depicts an alternative dissecting balloon catheter that may be readily incorporated into the instrument of FIG. 1; and

FIG. 8 depicts a second alternative dissecting balloon catheter that may be readily incorporated into the instrument of FIG. 1.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a surgeon, or other operator, grasping a surgical instrument having a distal surgical end effector. The term “proximal” refers to the position of an element arranged closer to the surgeon, and the term “distal” refers to the position of an element arranged closer to the surgical end effector of the surgical instrument and further away from the surgeon. Moreover, to the extent that spatial terms such as “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

As used herein, the terms “about” and “approximately” for any numerical values or ranges indicate a suitable dimensional tolerance that allows the part or collection of components to function for its intended purpose as described herein.

I. Exemplary Instrument for a Prolapsed Nasal Valve Procedure

As mentioned above, in some instances where a patient experiences a prolapsed nasal valve, it may be desirable to insert an implant within the interior of the lateral wall of the nose (e.g., the portion of the lateral wall not exposed medially to the nasal cavity or laterally to the external environment) for structural support. However, accessing the interior portion of the lateral wall of the nose for purposes of implant insertion may be difficult. For instance, an implant may not have the suitable structural integrity to initially form an incision and advancement within the lateral wall of the nose in order to access the interior portion of the lateral wall.

Additionally, even after the interior of the lateral wall of the nose is accessed, if the accessed area within the lateral wall is not of sufficient size to accommodate placement of an implant, suitable insertion of an implant still may not be possible. For instance, if a newly accessed area within the interior of the lateral wall forms an opening that is not large enough to accommodate insertion of the implant, the implant may not have the suitable structural integrity to further open the newly formed incision and/or accessed area within the interior of the lateral wall. In such assistances, if the operator attempted to insert the implant into the newly formed incision and further into the newly accessed area within the interior of the lateral wall, the implant may undesirably buckle, crumple, or otherwise deform due to contact with adjacent anatomy defining the newly formed opening.

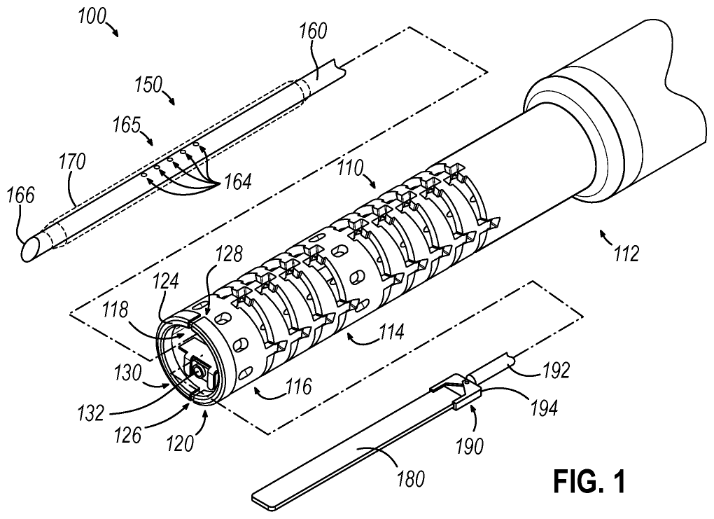

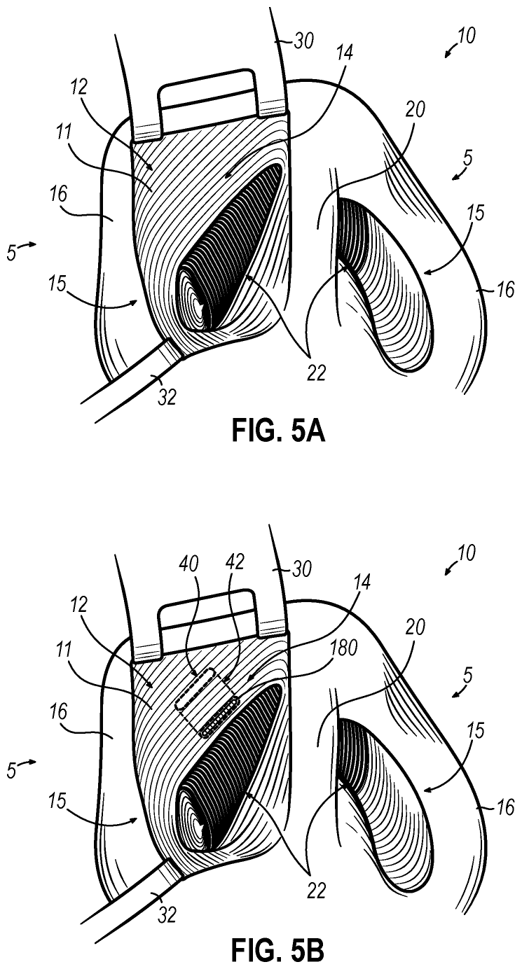

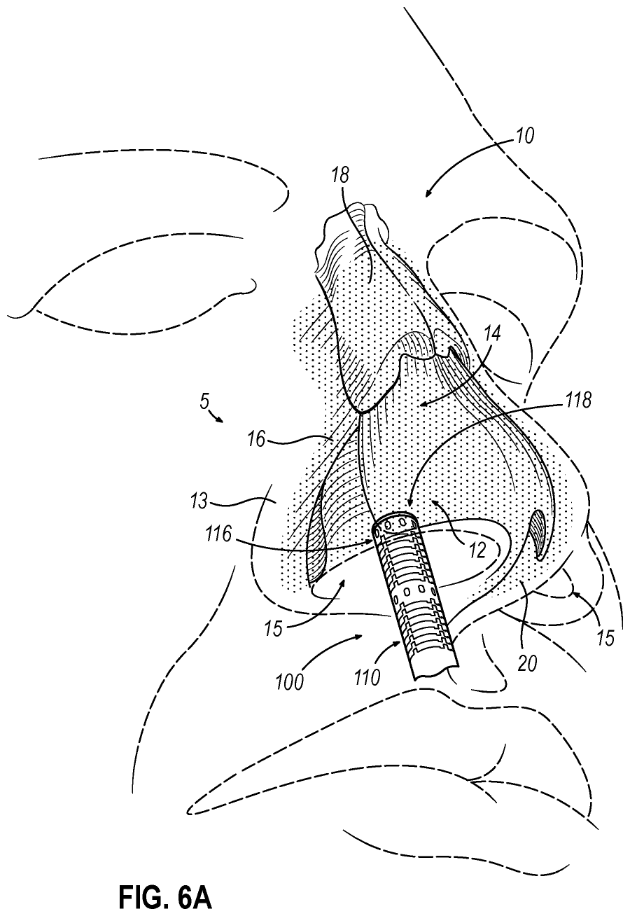

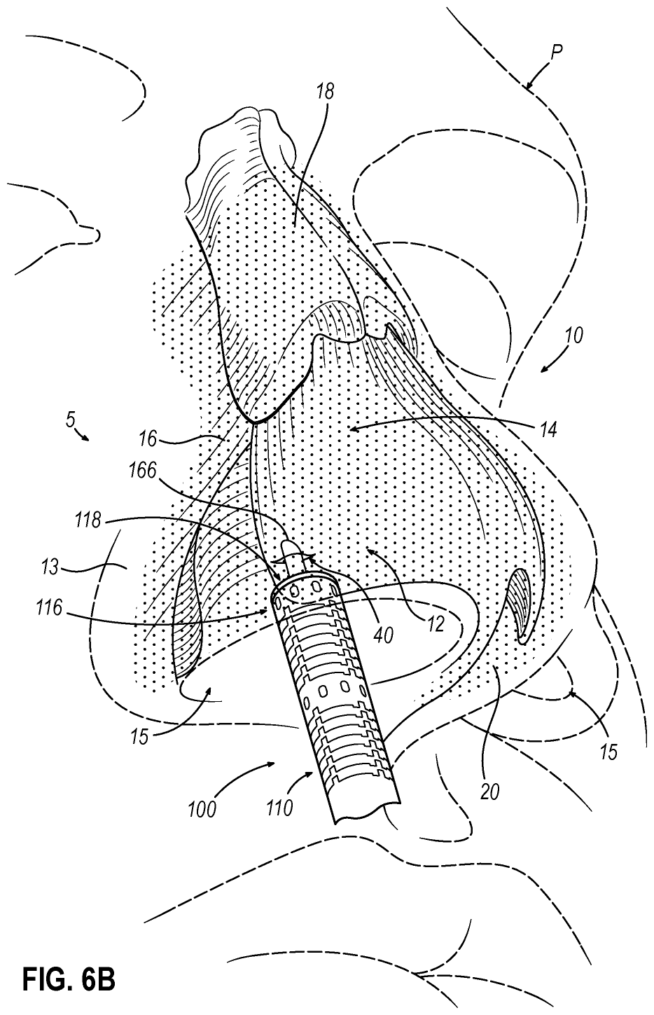

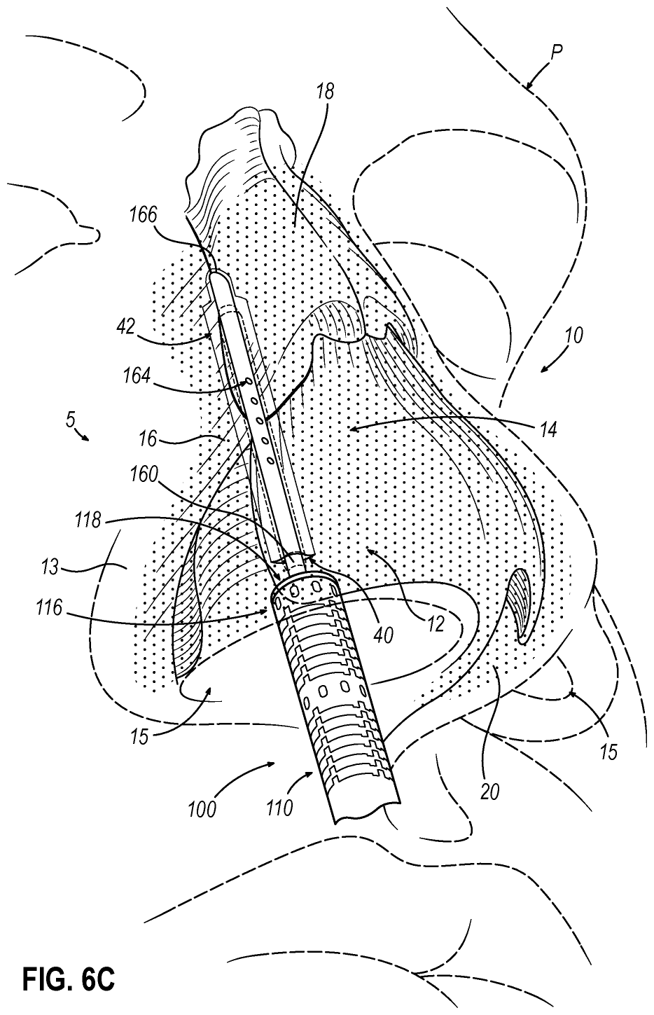

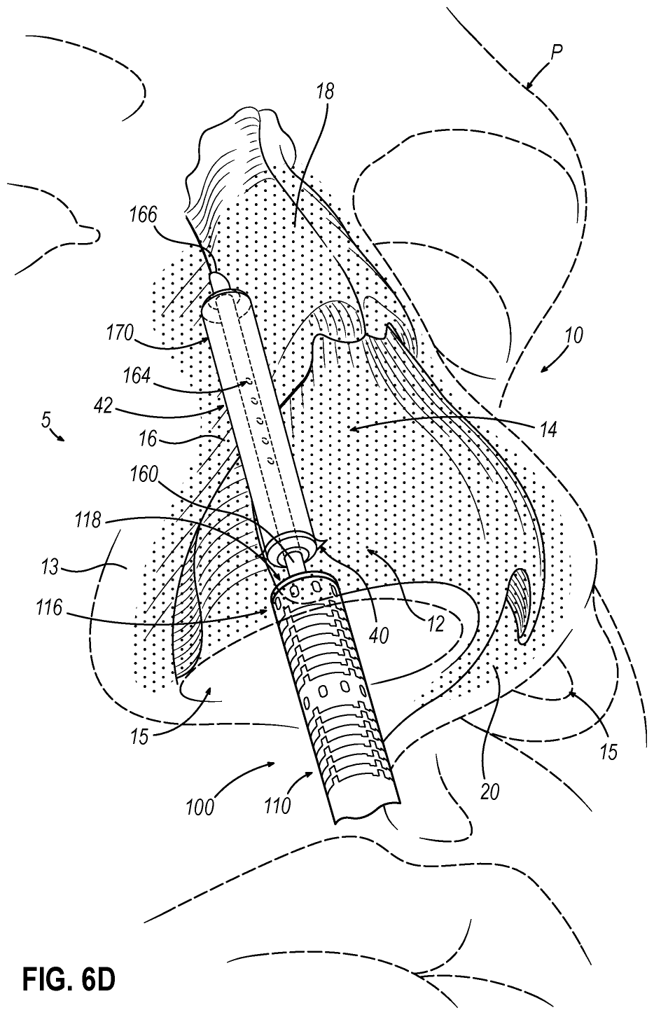

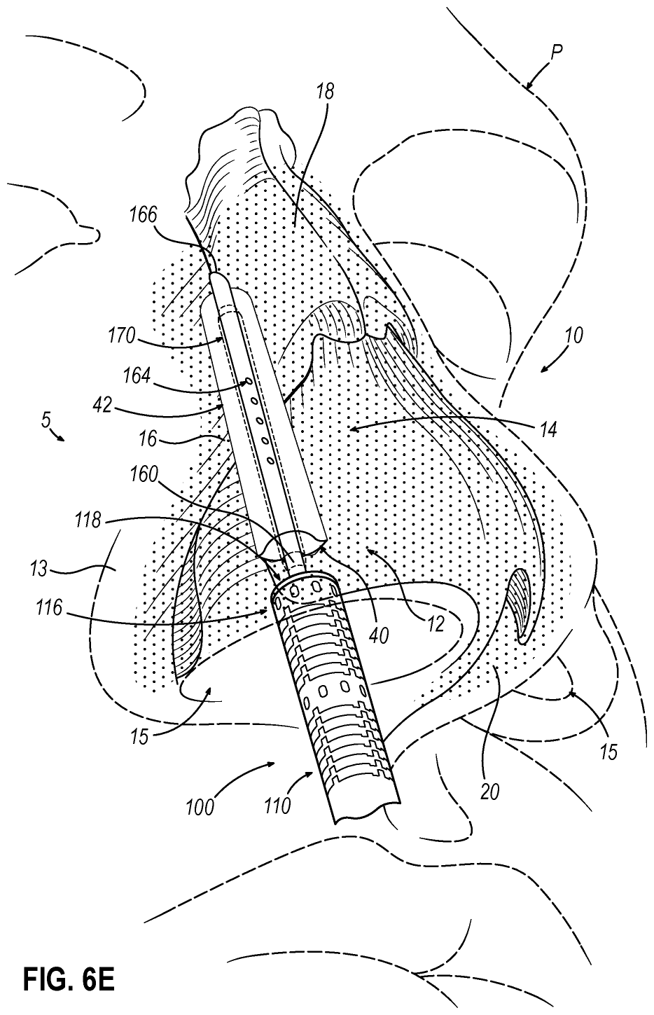

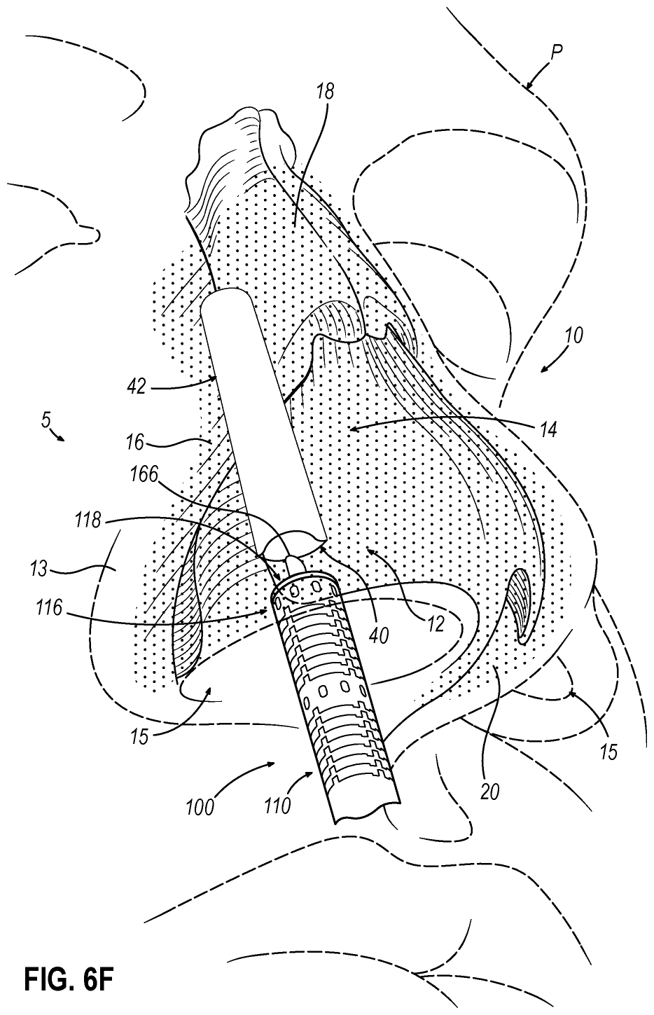

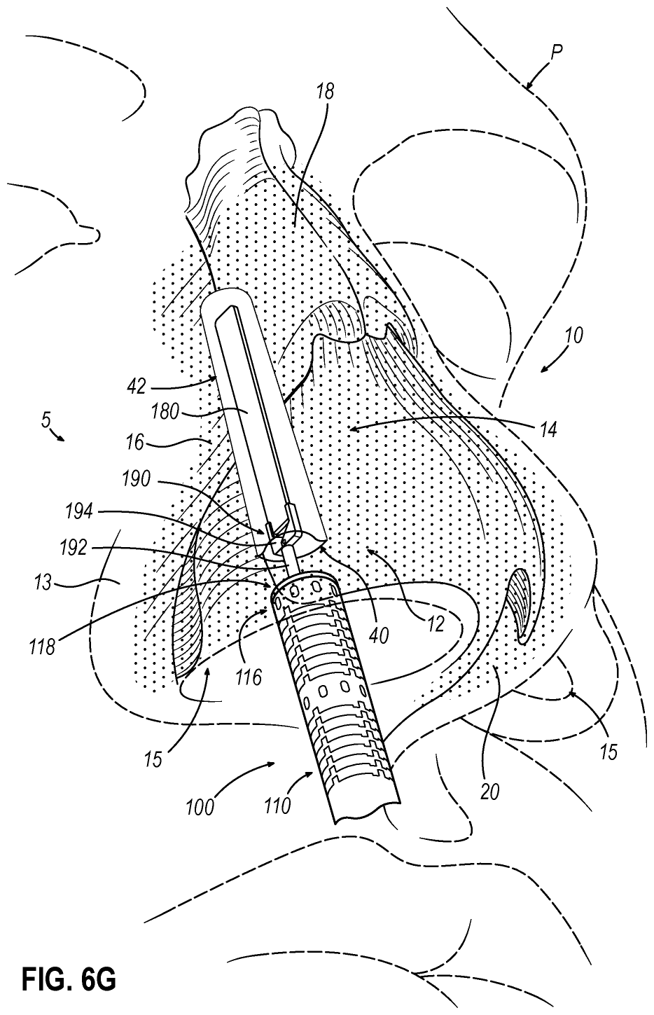

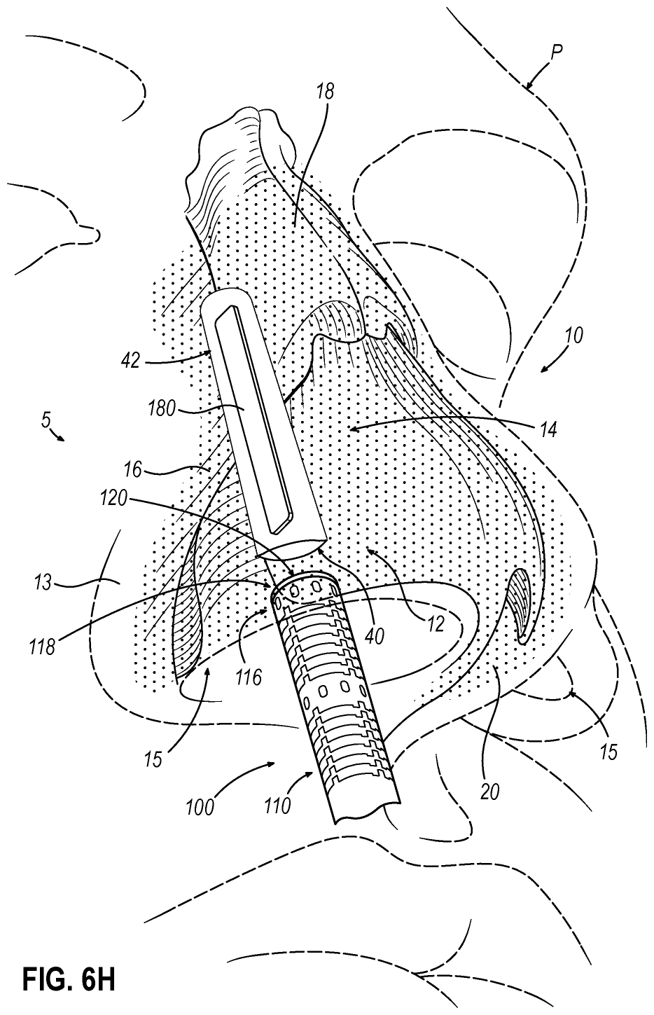

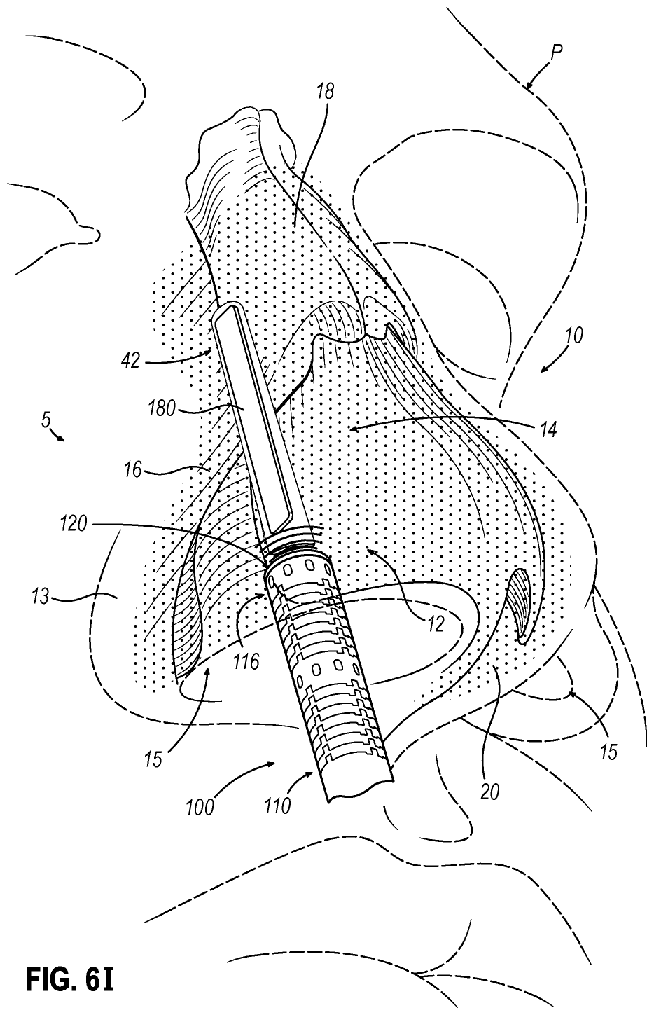

Therefore, it may be desirable to have an instrument configured to access the interior of a lateral wall (5) (see FIGS. 5A-5B) of a patient's nose (10), create an elevated pocket (42) (see FIGS. 5B and 6D-6I) within the interior of the lateral wall (5), and place an implant (180) within the newly created elevated pocket (42). FIG. 1 shows the distal end of an instrument (100) configured to perform such a procedure. Instrument (100) includes a guide catheter assembly (110), a dissecting balloon catheter (150), and polymer plate implant (180) selectively coupled to a deployment mechanism (190). As will be described in greater detail below, guide catheter assembly (110) slidably contains dissecting balloon catheter (150), polymer plate implant (180), and deployment mechanism (190) such that (A) dissecting balloon catheter (150) may initially access the interior of lateral wall (5) and create an elevated pocket (42) therewithin, and (B) deployment mechanism (190) may suitably deploy polymer plate implant (180) within the newly created elevated pocket (42) in order to structurally support lateral wall (5) of nose (10). As will also be described in greater detail below, polymer plate implant (180) may have sufficient characteristics in order suitably support a lateral wall (5) of the nose (10) in order to prevent lateral wall (5) from undesirably prolapsing toward the septum (20) during nasal breathing.

A. Exemplary Guide Catheter Assembly

Guide catheter assembly (110) includes a rigid proximal portion (112), a flexible portion (114) distal to rigid portion (112), and an open distal tip (116). Guide catheter assembly (110) may have any suitable length as would be apparent to one skilled in the art in view of the teachings herein. Guide catheter assembly (110) defines a working channel (118) that extends through rigid proximal portion (112) all the way to open distal tip (116). Dissecting balloon assembly (150), implant plate (180), and deployment mechanism (190) may all be slidably housed within working channel (118) such that suitable portions of a dissecting balloon assembly (150) may actuate distally past open distal tip (116); and selected portions of deployment mechanism (190) may actuate distally past open distal tip (116) in order to suitably deploy polymer plate implant (180) in accordance with the description herein.

A proximal end of rigid proximal portion (112) may include an open proximal end such that other suitable portions of instrument (100) may be manipulated, controlled, and actuated relative to guide catheter assembly (110). For example, rigid proximal portion (112) may be coupled to a handle (not shown) having various actuating bodies and buttons and/or other user input features configured to suitably actuate, activate/deactivate, or otherwise control other suitable components of instrument (100) as would be apparent to one skilled in the art in view of the teachings herein.

A pull-wire (not shown) may be coupled with flexible portion (114) and/or distal tip (116) and extend proximally along rigid proximal portion (112). Such a pull-wire may be selectively retracted proximally in order to bend flexible portion (114) and deflect open distal tip (116) laterally away from a longitudinal axis of rigid proximal portion (112). Therefore, such a pull-wire may impart steerability of guide catheter assembly (110). Such a pull-wire may be attached to any suitable control mechanism for actuating the pull-wire relative to the rest of guide catheter assembly (110) as would be apparent to one skilled in the art in view of the teachings herein. By way of example only, such steerability of guide catheter assembly (110) may be provided in accordance with at least some of the teachings of U.S. Pub. No. 2021/0361912, entitled “Shaft Deflection Control Assembly for ENT Guide Instrument,” published Nov. 25, 2021, the disclosure of which is incorporated by reference herein, in its entirety.

It should be understood that in some instances, flexible portion (114) may not be present, such that rigid proximal portion (112) extends directly into open distal tip (116). In such instances, guide catheter assembly (110) may not have any steerability. Guide catheter assembly (110) may thus be rigid along the entire shaft length of guide catheter assembly. Alternatively, flexible portion (114) may be replaced with a malleable region and/or may have any other suitable properties.

An arcuate electrode assembly (120) and a visualization assembly (130) are fixed to guide catheter assembly (110) near open distal tip (116). Arcuate electrode assembly (120) includes a first arched electrode body (122) and a second arched electrode body (124) attached at the circumferential rim of open distal tip (116) defining working channel (118). Each electrode body (122, 124) is coupled with a corresponding one or more wire(s), trace(s), and/or other conductive element(s) that electrically couple electrode bodies (122, 124) with an RF generator (not shown). Such an RF generator may selectively activate arcuate electrode assembly (120) in accordance with the description herein.

In the present example, first electrode body (122) is configured to apply RF energy at a first polarity; while second electrode body (124) is configured to apply RF energy at a second polarity. Electrode bodies (122, 124) thus serve as electrodes that are operable to apply bipolar RF energy to tissue contacting electrode bodies (122, 124). By way of example only, first electrode body (122) may serve as an active electrode while second electrode body (124) may serve as a return electrode. By applying bipolar RF energy to tissue, electrode bodies (122, 124) may be configured to seal tissue as would be apparent to one skilled in the art in view of the teachings herein.

Open distal tip (116) may be formed of an electrically insulative material, such as a plastic material, that prevents short circuiting between electrode bodies (122, 124) while mechanically securing electrode bodies (122, 124) relative to each other in a spaced-apart relationship. With this spacing maintained by open distal tip (116), a first angular gap (126) is defined between respective first free ends of electrode bodies (122, 124); while a second angular gap (128) is defined between respective second free ends of electrode bodies (122, 124). Gaps (126, 128) are angularly offset from each other by approximately 180 degrees in the present example, although any suitable spatial relationship between gaps (126, 128) may be used as would be apparent to one skilled in the art in view of the teachings herein.

Electrode bodies (122, 124) may be formed of any suitable material or combination of materials as would be apparent to one skilled in the art in view of the teachings herein. Additionally, while in the current example electrode bodies (122,124) have a circumferential shape surrounding a portion of distal tip (116) defining working channel (118), electrode bodies (112, 124) may have any suitable shape, geometry, orientation, etc., as would be apparent to one skilled in the art in view of the teachings herein.

Visualization assembly (130) includes a camera (132) mounted within working channel (118) at open distal tip (116) such that camera (132) is configured to capture images near open distal tip (116). Camera (132) may allow an operator to visually confirm placement of open distal tip (116), dissecting balloon catheter (150), and polymer plate implant (180) during exemplary use. While not shown, visualization assembly (130) may include a communication wire, trace, etc., extending proximally from camera (132) within working channel (118). Communication wire may connect camera (132) with a suitable display screen so an operator may see images captured by camera (132). Visualization assembly (130) may also include one or LEDs, optical fibers, and/or other elements that are operable to illuminate the field of view of camera (132).

B. Exemplary Dissecting Balloon Catheters

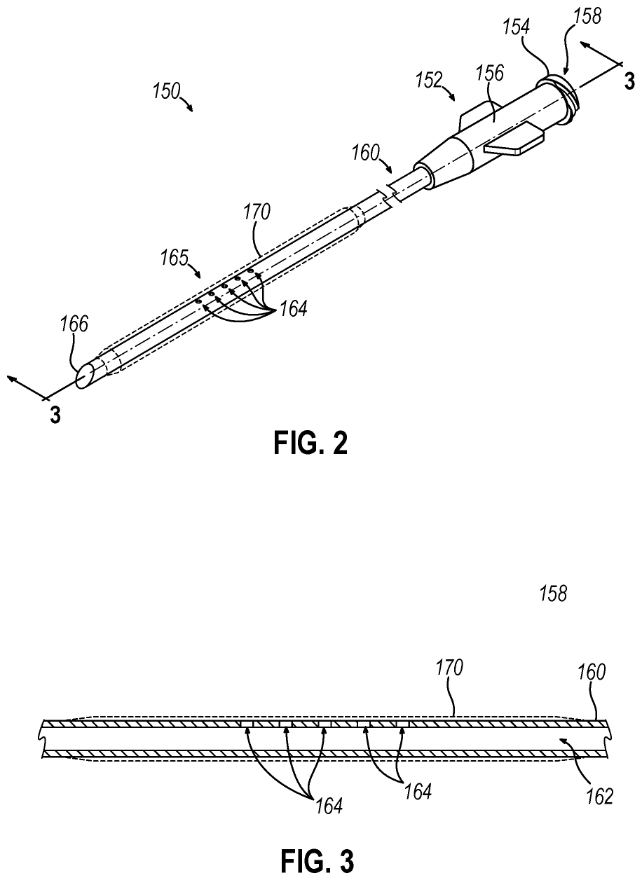

FIGS. 2-4B show an exemplary dissecting balloon catheter (150). As mentioned above, and as will be described in greater detail below, dissecting balloon catheter (150) is configured to initially access the interior of lateral wall (5) of nose (10) and create an elevated pocket (42) therewithin. As best shown in FIG. 2, dissecting balloon catheter (150) includes a proximal body (152), an elongated stylet shaft (160) extending distally from proximal body (152) and terminating into a sharp, tissue-penetrating distal tip (166), and an expanding body (170) attached to elongated stylet shaft (160) at a distal portion (165) dissecting balloon catheter (150). It should be understood that elongated stylet shaft (160) may have any suitable length as would be apparent to one skilled in the art in view of the teaching herein.

As best seen in FIG. 2, proximal body (152) includes a fluid coupling feature (154) and griping body (156). In some instances, when instrument (100) is assembled, proximal body (152) may be located proximally to the open proximal end of guide catheter assembly (110). Therefore, an operator may grasp proximal body (152) in order to actuate dissecting balloon catheter (150) longitudinally relative to guide catheter assembly (110). In some instances, proximal body (152) may be slidably/rotatably attached to another handle (not shown) fixed to the proximal end of guide catheter assembly (110). In such instances, proximal body (152) may be able to rotate about its own longitudinal axis, and translate, relative to the handle (not shown) fixed to the proximal end of guide catheter assembly (110).

Gripping body (156) provides a handle for an operator to grasp and control dissecting balloon catheter (150) in accordance with the description herein. Therefore, an operator may longitudinally actuate gripping body (156) in order to advance and retract expanding body (170) and penetrating distal tip (166) relative to open distal tip (116) of guide catheter assembly (110). Additionally, an operator may grasp gripping body (156) and rotate gripping body (156) about its own longitudinal axis in order to rotate expanding body (170) and penetrating distal tip (166) about their respective longitudinal axis.

Fluidic coupling feature (154) of proximal body (152) defines a proximal fluidic opening (158). Proximal fluidic opening (158) is in fluid communication with an inner lumen (162) (see FIG. 3) defined by elongated stylet shaft (160). Fluid coupling feature (154) is configured to couple with a fluid source (such as a syringe, pump, etc.) to establish fluid communication between the fluid source and an inner lumen (162) (see FIG. 3). As will be described in greater detail below, inner lumen (162) (see FIG. 3) is configured to transmit fluid to/from the fluid source to deflate/inflate expanding body (170) via at least one inflation port (164) (see FIG. 3). Fluidic coupling feature (154) may include any suitable structures to couple with the fluid source as would be apparent to one skilled in the art in view of the teachings herein. For example, fluidic coupling feature (154) may include a Luer coupling.

Distal portion (165) of dissecting balloon catheter (150), including distal tip (166) and expanding body (170), is dimensioned with a very thin outer profile such that corresponding portions of elongated stylet shaft (160) and expanding body (170) may be inserted within the interior of lateral wall (5) of nose (10). Penetrating distal tip (166) is sufficiently sharp in order to create an initial incision (40) (see FIGS. 5B and 6B) on an external nasal cavity surface (11) of lateral wall (5) (see FIGS. 5A-5B). Further, penetrating distal tip (166) is also sufficiently sharp in order dissect portions of the interior of lateral wall (5) elevated superiorly from initial incision, thereby allowing an operator to further advance the distal portion (165) of elongated stylet shaft (160) within the interior of lateral wall (5) of nose (10) to a desired location (see FIG. 6C).

In the current example, penetrating distal tip (166) includes a tapered tip. However, penetrating distal tip (166) may include any suitable shaped distal tip as would be apparent to one skilled in the art in view of the teachings herein. For example, penetrating distal tip (166) may include a conical tip having a centered piercing edge. Additionally, penetrating distal tip (166) may extend any suitable length away from the distal end of expanding body (170) as would be apparent to one skilled in the art in view of the teachings herein.

Elongated stylet shaft (160) is formed of a material having sufficient column strength such that as the distal portion (165) is advance within the interior of lateral wall (5) in accordance with the description herein, elongated stylet shaft (160) maintains its structural integrity. Therefore, the elongated stylet shaft (160) has sufficient column strength to allow the operator to have suitable control of elongated stylet shaft (160) during advancement within lateral wall (5) in accordance with the description herein. In other words, elongated stylet shaft (160) has sufficient column strength such that shaft (160) does not buckle, undesirably bend, or otherwise undesirably deform in response to penetrating distal tip (166) dissecting the interior of lateral wall (5) during advancement distal portion (165) within lateral wall (5).

Expanding member (170) is attached to a distal portion of elongated stylet shaft (160). In the current example, expanding member (170) is fixed to selected portions of elongated stylet shaft (160) at a proximal end and a distal end of expanding member (170). However, it should be understood that expanding member (170) may attach to any suitable portion of elongated stylet shaft (160) as would be apparent to one skilled in the art in view of the teachings herein.

Expanding member (170) surrounds a portion of elongated stylet shaft (160) defining inflation ports (164) such that an interior of expanding member (170) is in fluid communication with inner lumen (162) (see FIG. 3) of elongated stylet shaft (160). In the current example, expanding member (170) is a balloon. Any suitable material may be used to form expanding member (170) as would be apparent to one skilled in the art in view of the teachings herein.

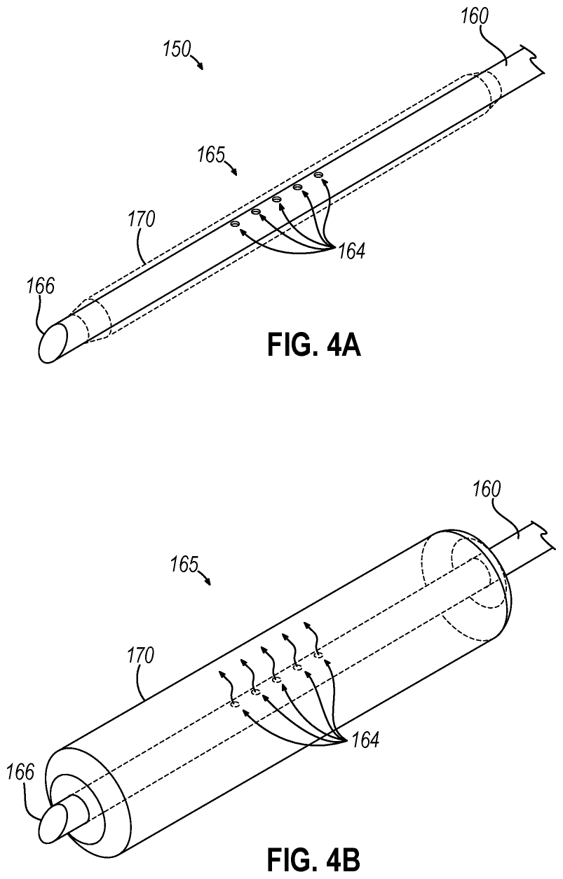

As mentioned above, inner lumen (162) (see FIG. 3) is in fluid communication with inflation ports (164) and extends proximally from distal portion (165) all the way to proximal fluid opening (158) of proximal body (152). Therefore, an operator may selectively inflate expanding number (170) from a deflated configuration (see FIG. 4A) into an inflated configuration (see FIG. 4B) by transmitting fluid through inner lumen (162), through inflation ports (164) and within the confines of expanding member (170). Similarly, the operator may selectively deflate expanding number (170) by suctioning fluid from expanding number (170), through inflation ports (164), and proximally through inner lumen (162). As will be described in greater detail below, while distal portion (165) is suitably inserted within the interior of lateral wall (5) of a nose (10), an operator may selectively inflate and deflate expanding member (170) in order to create an elevated pocket (42) within the interior of lateral wall (5).

In the current example, expanding member (170) includes a balloon configured to transition between the deflated configuration and the expanded configuration via any suitable fluid provided via a fluid source. However, this is merely optional, as any suitable expanding member may be used as would be apparent to one skilled in the art in view of the teachings herein. In some versions, a liquid such as saline, etc., is used to inflate expanding member (170). In some other versions, a gas such as air, etc., is used to inflate expanding member.

It should be understood that expanding member (170) includes a suitable shape, size, geometry, and material such that as distal portion (165) of dissecting balloon catheter (150) is inserted/retracted within lateral wall (5) of the nose (10) in accordance with the description herein, expanding member (170) may also be inserted/restricted within lateral wall (50) of the nose (10) in the deflated configuration without damaging or injuring adjacent tissue and other anatomical structures. In other words, the presence of expanding member (170) on distal portion (165) of dissecting balloon catheter (150) does not inhibit, or otherwise undesirably affect, the insertion/retraction of distal portion (165) within lateral wall (5) of the nose (10).

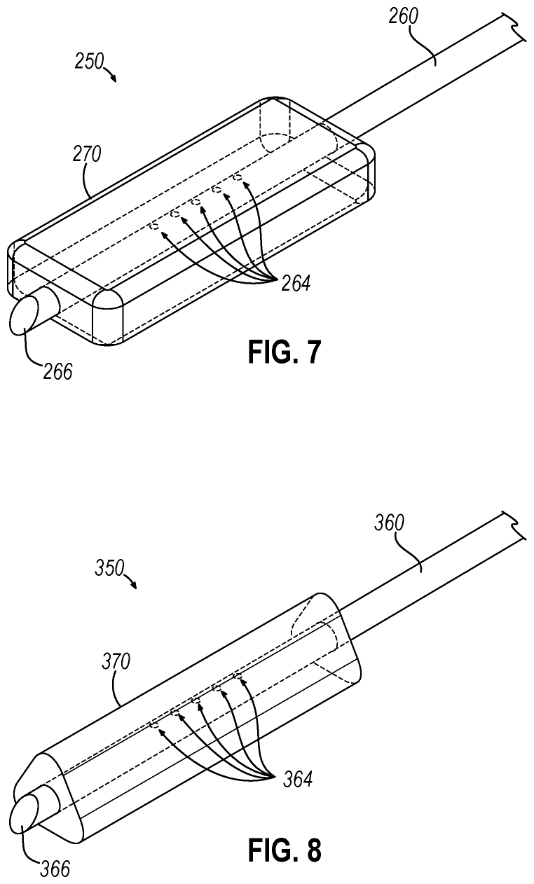

In the current example, expanding member (170) of dissecting balloon catheter (150) is substantially cylindrical in shape when in the inflated configuration (see FIG. 4B). However, it should be understood that expanding member (170) may have any suitable geometry when in the inflated configuration, as would be apparent to one skilled in the art in view of the teachings herein. For instance, FIG. 7 shows an exemplary alternative dissecting balloon catheter (250) that may be readily incorporated into instrument (10) described herein, in replacement of dissecting balloon catheter (150). Dissecting balloon catheter (250) is substantially similar to dissecting balloon catheter (150) described above, with difference elaborated below. Dissecting balloon catheter (250) includes a proximal body (not shown), an elongated stylet shaft (260) defining an inner lumen (not shown) and a plurality of inflation ports (264), a penetrating distal tip (266), and an expanding body (270), which are substantially similar to proximal body (152), elongated style shaft (160), inner lumen (162), plurality of inflation ports (164), penetrating distal tip (166), and expanding body (170), with differences elaborated below.

In particular, the geometry of expanding body (270) is a substantially rectangular shape, rather than a substantially cylindrical shape like expanding body (170) described above. Therefore, expanding body (270) may be inflated to expand an opening within the interior of lateral wall (5) of the nose (10), such that the newly expanded opening within the interior of lateral wall (5) takes on a rectangular geometrical profile similar to the shape of expanding body (270). Therefore, the geometry of expanding body (270) may be modified to better accommodate the insertion of implant (180) in accordance with the description herein.

FIG. 8 shows another exemplary alternative dissecting balloon catheter (350) that may be readily incorporated into instrument (10) described herein, in replacement of dissecting balloon catheter (150). Dissecting balloon catheter (350) is substantially similar to dissecting balloon catheter (150) described above, with difference elaborated below. Dissecting balloon catheter (350) includes a proximal body (not shown), an elongated stylet shaft (360) defining an inner lumen (not shown) and a plurality of inflation ports (364), a penetrating distal tip (366), and an expanding body (370), which are substantially similar to proximal body (152), elongated style shaft (160), inner lumen (162), plurality of inflation ports (164), penetrating distal tip (166), and expanding body (170), with differences elaborated below.

In particular, the geometry of expanding body (370) is a substantially triangular shape, rather than a substantially cylindrical shape like expanding body (170) described above. Therefore, expanding body (370) may be inflated to expand an opening within the interior of lateral wall (5) of the nose (10), such that the newly expanded opening within the interior of lateral wall (5) takes on a triangular geometrical profile similar to the shape of expanding body (370). Therefore, the geometry of expanding body (370) may be modified to better accommodate the insertion of implant (180) in accordance with the description herein.

While expanding body (170, 270, 370) is described as having cylindrical, rectangular, and triangular shapes, any other suitable geometrical profile may be used as would be apparent to one skilled in the art in view of the teachings herein.

C. Exemplary Polymer Plate Implant and Deployment Mechanism

FIG. 1 shows exemplary polymer plate implant (180) selectively attached to deployment mechanism (190). As mentioned above, deployment mechanism (190) may suitably deploy polymer plate implant (180) within the newly created elevated pocket (42) in order to structurally support lateral wall (5) of nose (10). Additionally, as mentioned above, polymer plate implant (180) may have sufficient characteristics in order to suitably support a lateral wall (5) of the nose (10) to thereby inhibit lateral wall (5) from undesirably prolapsing toward the septum (20) during nasal respiration.

Polymer plate implant (180) is formed from a suitable biocompatible material such that implant (180) may be permanently implanted within lateral wall (5) of nose (10). In some instances, polymer plate implant (180) may be configured to promote tissue growth on the exterior of implant (180) such that implant (180) becomes relatively fixed within lateral wall (5) after elevated pocket (42) completely heals. In some instances, polymer plate implant (180) may have a coating of a suitable therapeutic agent, such that after implant (180) is inserted within elevated pocket (42) in accordance with the description herein, the therapeutic agent interacts with the surrounding anatomy. Any suitable therapeutic agent may be used as would be apparent to one skilled in the art in view of the teachings herein. In some instances, implant (180) may be bioabsorbable, so when elevated pocket (42) heals, implant (180) is absorbed over a suitable period of time.

Polymer plate implant (180) is formed of a material having sufficient rigidity to support the lateral wall (5) of a nose (10) after implantation, thereby inhibiting lateral wall (5) from undesirably prolapsing toward the septum (20) during nasal respiration. Additionally, implant (180) is sufficiently pliable such that implant (180) is configured to suitably conform to the preexisting profile of the lateral wall (5) after implant (180) is completely implanted in accordance with the description herein. In other words, once implant (180) is inserted within the lateral wall (5) in accordance with the description herein, implant (180) may structurally bolster lateral wall (5) to inhibit nasal prolapse, while also complying with the intended shape of lateral wall (5). Polymer plate implant (180) may be formed of any suitable material(s) as would be apparent to one skilled in the art in view of the teachings herein. In the current example, polymer plate implant (180) is generally rectangular shape. However, implant (180) may have any suitable shape as would be apparent to one skilled in the art in view of the teachings herein.

Deployment mechanism (190) includes an elongated shaft assembly (192) terminating distally into a grasp and release assembly (194). Elongated shaft assembly (192) extends proximally through open proximal end of guide catheter assembly (110) such that an operator may grasp a proximal portion of elongated shaft assembly (192) in order to actuate deployment mechanism (190) relative to guide catheter assembly (110). Elongated shaft assembly (192) may have any suitable length as would be apparent to one skilled in the art in view of the teachings herein. While not shown, deployment mechanism (190) may include a proximal handle (not shown) attached to the proximal end of elongated shaft assembly (192). Such a proximal handle (not shown) may include various features allowing an operator to manipulate grasp and release assembly (194) to selectively manipulate and actuate implant (180) in accordance with the description herein.

In the current example, grasp and release assembly (194) includes a pair of jaws configured to pivot relative to each other in order to suitably manipulate implant (180). Grasp and release assembly (194) is configured to suitably control implant (180) such that an operator may actuate a proximal end of elongated shaft assembly (192) in order to actuate implant (180) when under the control of grasp and release assembly (194). Grasp and release assembly (194) may be configured to suitably grasp implant (180), such that as implant (180) is being inserted within elevated pocket (42) in accordance with the description herein, incidental contact between implant (180) and adjacent anatomy of the patient does not undesirably affect the ability of grasp and release assembly (194) to control and manipulate implant (180) in accordance with the description herein. Grasp and release assembly (194) is also configured to selectively release or otherwise decouple from implant (180), such that deployment mechanism (190) may move relative to implant (180) after releasing implant (180) in accordance with the description herein.

While in the current example, grasp and release assembly (194) includes a pair of jaws, any suitable structure may be used to form grasp and release assembly (194) as would be apparent to one skilled in the art in view of the teachings herein. For example, grasp and release assembly (194) may include an adhesive that selectively couples the distal end of elongated shaft assembly (192) with implant (180).

II. Example Method of Performing a Prolapsed Nasal Valve Procedure

FIGS. 5A-5B and FIGS. 6A-6I show an exemplary method of performing a prolapsed nasal valve procedure utilizing instrument (100) described above. First, as shown in FIG. 5A, the operator may prepare to access the nasal cavity (15) of a patient by suitably expanding portions of nose (10) defining nasal cavity (15) via elevators (30, 32). It should be understood that any suitable tools may be used to expand the opening of nasal cavity (15) as would be apparent to one skilled in the art in view of the teachings herein. Additionally, in some instance, expanding the opening of nasal cavity (15) may not be required, such that elevators (30, 32) are not used.

Next, was shown in FIG. 6A, the operator may advance guide catheter assembly (110) such that open distal tip (116) is positioned within nasal cavity (15) or directly adjacent to nasal cavity (15). Additionally, working channel (118) is aligned with an interior surface (11) (see FIGS. 5A-5B) of lateral wall (5). The operator may utilize camera (132) of visualization assembly (130) to visually confirm proper placement of guide catheter assembly (110).

In the present example, at the moment shown in FIG. 6A, distal portion (165) of dissecting balloon catheter (150), polymer plate implant (180), and suitable portions of deployment mechanism (190) are all slidably contained within working channel (118) of guide catheter assembly (110). However, in some instances, dissecting balloon catheter (150), polymer plate implant (180), and suitable portions of deployment mechanism (190) may be initially introduced through the open proximal end of guide catheter assembly (110) immediately prior to their use in accordance with the description herein, or at any other suitable time as would be apparent to one skilled in the art in view of the teachings herein.

In the present example, open distal tip (116) is positioned to be directly adjacent to a portion of interior surface (11) adjacent to lower lateral cartilage (12). However, the operator may advance open distal tip (116) within nasal cavity (15) and directly adjacent to any suitable portion of interior surface (11) as would be apparent to one skilled in the art in view of the teachings herein.

Next, as shown in FIG. 6B, the operator may advance penetrating distal tip (166) of dissecting balloon assembly (150) distally past open distal tip (116) of guide catheter assembly (110). Additionally, the operator may advance penetrating distal tip (166) in order to form an initial incision (40) with a portion of lateral mucosal tissue (16) forming interior surface (11) (see FIG. 5B) of lateral wall (5). At the moment shown in FIG. 6B, the penetrating distal tip (166) is within the interior of lateral wall (5) of nose (10) such that penetrating distal tip (166) is between adjacent portions of interior surface (11) (see FIGS. 5A-5B) and an exterior surface (13) of lateral wall (5).

The operator may utilize camera (132) of visualization assembly (130) to visually confirm proper placement of guide catheter assembly (110). During this advancement, the operator may grasp or otherwise control suitable portions of the nose (10) or other anatomy of the patient in order to further control and guide the placement of penetrating distal tip (166) shown between FIGS. 6A-6B.

Next, as shown in FIG. 6C, the operator may advance penetrating distal tip (166) superiorly within the interior of lateral wall (5) of nose (10) in order to dissect corresponding mucosal tissue (16) and initially create an elevated pocket (42) along the interior of lateral wall (5). Penetrating distal tip (166) may be advanced through mucosal tissue (16) to any suitable location along lateral wall (5) as would be apparent to one skilled in the art in view of the teachings herein. In the current example, penetrating distal tip (166) is advanced to a position adjacent to nasal bone (18).

During advancement of penetrating distal tip (166), elongated stylet shaft (160) has sufficient column strength to allow the operator to maintain suitable control of dissecting balloon catheter (150) while distal tip (166) dissects mucosal tissue (16) within lateral wall (5). In other words, elongated stylet shaft (160) is sufficiently rigid to inhibit undesirable bending or buckling in response to distal tip (166) encountering and separating mucosal tissue (16) while creating corresponding portions of elevated pocket (42). During this advancement, the operator may grasp or otherwise control suitable portions of the nose (10) or other anatomy of the patient in order to further control and guide the placement of penetrating distal tip (166) shown between FIGS. 6B-6C.

While a distal portion of expanding member (170) is housed within the initially created elevated pocket (42), a portion of expanding member (170) may extend proximally from initial incision (40) such that the entirety of expanding number (170) is not completely housed within elevated pocket (42). The operator may utilize camera (132) of visualization assembly (130) to visually confirm proper placement of expanding number (170). In some instances, penetrating distal tip (166) may only pierce and dissect portions of mucosal tissue (16) forming lateral wall (5) of nose (10) in order to form initial incision (40) and elevated pocket (42). In other instances, penetrating distal tip (166) may pierce suitable portions of lower lateral cartilage (12) or upper lateral cartilage (14) while initially forming incision (40) and elevated pocket (42).

With elevated pocket (42) initially formed via advancement of penetrating distal tip (166), the size of elevated pocket (42) may still not be sufficient to accommodate placement of implant (180) within pocket (42). Therefore, it may be desirable to further expand elevated pocket (42) in order to create sufficient space for receiving implant (180). To that end, as shown in FIG. 6D, the operator may inflate expanding member (170) into the expanded configuration in accordance with the description above. Inflation of expanding member (170) forces contact between an exterior surface of expanding member (170) and the newly formed elevated pocket (42) such that the anatomy defining elevated pocket (42) is expanded, thereby increasing the area of elevated pocket (42). Since a portion of expanding member (170) extends proximally from initial incision (40) in this example, initial incision (40) is also expanded in order to accommodate the insertion of implant (180) into elevated pocket (140). The operator may utilize camera (132) of visualization assembly (130) to visually confirm expansion of initial incision (40). The operator may leave expanding member (170) in the inflated configuration for a suitable period of time such that initial incision (40) and elevated pocket (42) retain a large enough opening to receive implant (180) in accordance with the description herein. In some scenarios, the operator may repeatedly inflate and deflate expanding member (170) to further achieve dilation of elevated pocket (42).

Next, as shown in FIG. 6E, the operator may deflate expanding member (170) in accordance with the description above. Once expanded member (170) is deflated (170), elevated pocket (42) and initial incision (40) are larger compared to the moment shown in FIG. 6C. This increase is size of elevated pocket (42) and initial incision (40) may accommodate the insertion of implant (180) into elevated pocket (42). The operator may utilize camera (132) of visualization assembly (130) to visually confirm the size of initial incision (40) and elevated pocket (42).

Next, as shown in FIG. 6F, dissecting balloon catheter (150) may be retracted proximally back into the confines of guide catheter assembly (110), which may be visually confirmed by utilization of camer (132) of visualization assembly (130). With dissecting balloon catheter (150) retracted proximally, the operator may advance implant (180) through the enlarged initial incision (40) into elevated pocket (42) with deployment mechanism (190) in accordance with the description herein, as shown in FIG. 6G. Since initial incision (40) and elevated pocket (42) are enlarged, implant (180) may be more easily placed within elevated pocket (42); as compared to an alternative scenario where pocket (42) would not have been enlarged by expanding member (170). If the structure of implant (180) were required to further enlarge pocket (42), implant (180) may undesirably deform, which may lead to various undesirable consequences, such as not being able to insert implant (180) within the desired location of pocket (42).

Next, as shown in FIG. 6H, the operator may release implant (180) from deployment mechanism (190) and retract deployment mechanism (190) back within the confines of guide catheter assembly (110). Therefore, implant (180) may be placed within the desired location within elevated pocket (42). With implant (180) inserted within the desired location of elevated pocket (42), implant (180) may be able to structurally support lateral wall (5) to inhibit prolapsing of lateral wall (5) toward septum (20) during nasal respiration. This may lead to various quality of life benefits to the patient in accordance with the description herein.

As shown in FIG. 6I, the operator may advance guide catheter assembly (110) to abut against the tissue adjacent to initial incision (40) and apply bipolar RF energy to tissue via electrode assembly (120) in accordance with the description herein, in order to seal initial incision (40). Next, the operator may remove guide catheter assembly (110) from nasal cavity (15). After a suitable period of time, elevated pocket (42) may retract in size, thereby conforming to the shape of implant (180).

III. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A method of inserting an implant within a lateral wall of a nose of a patient, the method comprising: (a) forming an initial incision with a surface of the lateral wall of the nose; (b) advancing a stylet within the lateral wall of the nose, thereby creating a pocket within the lateral wall of the nose; (c) further advancing the stylet until an expanding body of the stylet is at least partially housed within the pocket of the lateral wall; (d) enlarging the expanding body of the stylet to thereby enlarge the pocket within the lateral wall; and (e) placing an implant within the enlarged pocket of the lateral wall.

### Example 2

The method of Example 1, wherein the expanding body comprises a balloon, wherein the stylet comprises an elongated shaft attached to the balloon.

### Example 3

The method of Example 2, wherein the elongated shaft defines an inflation port in fluid communication with the balloon.

### Example 4

The method of any one or more of Examples 2 through 3, wherein the balloon defines a cylindrical shape.

### Example 5

The method of any one or more of Examples 2 through 3, wherein the balloon defines a rectangular shape.

### Example 6

The method of any one or more of Examples 2 through 3, wherein the balloon defines a triangular shape.

### Example 7

The method of any one or more of Examples 1 through 6, wherein the initial incision is formed with a distal tip of the stylet.

### Example 8

The method of any one or more of Examples 1 through 7, wherein the surface of the lateral wall comprises an interior surface defining a nasal cavity.

### Example 9

The method of any one or more of Examples 1 through 8, further comprising advancing a guide shaft adjacent to the surface of the lateral all of the nose.

### Example 10

The method of Example 9, wherein the guide shaft comprises a working channel, wherein the stylet is slidably housed within the working channel.

### Example 11

The method of Example 10, further comprising advancing an electrode assembly of the guide shaft against the incision and applying RF energy to the incision via the electrode assembly.

### Example 12

The method of any one or more of Examples 1 through 11, further comprising advancing the implant within the pocket of the lateral wall with a deployment mechanism.

### Example 13

The method of Example 12, wherein the deployment mechanism comprises a pair of jaws, wherein advancing the implant further comprises grasping the implant with the pair of jaws.

### Example 14

The method of Example 13, wherein placing the implant within the pocket of the lateral wall further comprises releasing the implant from the pair of jaws.

### Example 15

The method of any one or more of Examples 1 through 14, wherein the implant comprises a polymer.

### Example 16

The method of any one or more of Examples 1 through 15, wherein the implant comprises a plate forming a rectangular shape.

### Example 17

The method of any one or more of Examples 1 through 16, wherein the implant further comprises a therapeutic agent.

### Example 18

The method of any one or more of Examples 1 through 17, wherein the implant comprises a bioabsorbable material.

### Example 19

A method of inserting an implant within a lateral wall of a nose of a patient, the method comprising: (a) forming a pocket within tissue in an interior portion of the lateral wall of the nose; (b) expanding the pocket within the interior portion of the lateral wall of the nose with a balloon; (c) inserting an implant within the expanded pocket.

### Example 20

A method of inserting an implant within a lateral wall of a nose of a patient, the method comprising: (a) penetrating the lateral wall of the nose to thereby access an interior lining of the lateral wall of the nose; (b) forming an expanded pocket within the interior lining of the lateral wall of the nose by inflating a balloon; (c) deflating the balloon; (d) removing the balloon from the pocket; and (e) inserting an implant within the pocket after the pocket has been expanded by the balloon.

### Example 21

An instrument for inserting an implant within the lateral wall of a nose of a patient, the instrument comprising: (a) a rigid stylet extending along a longitudinal axis, wherein the stylet comprises a piercing distal tip, wherein the stylet defines an inner lumen and at least one inflation port, wherein the piercing distal tip is configured to create an incision within the lateral wall of the nose; (b) a balloon coupled with the rigid stylet, wherein the balloon surrounds the at least one inflation port, wherein the balloon is configured to transition between a deflated configuration and an inflated configuration; and (c) an implant sized and configured for insertion in a tissue pocket created by the balloon.

IV. Miscellaneous

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A method of inserting an implant within a lateral wall of a nose of a patient, the method comprising:
(a) forming an initial incision with a surface of the lateral wall of the nose;
(b) advancing a stylet within the lateral wall of the nose, thereby creating a pocket within the lateral wall of the nose;
(c) further advancing the stylet until an expanding body of the stylet is at least partially housed within the pocket of the lateral wall;
(d) enlarging the expanding body of the stylet to thereby enlarge the pocket within the lateral wall; and
(e) placing an implant within the enlarged pocket of the lateral wall.

2. The method of claim 1, wherein the expanding body comprises a balloon, wherein the stylet comprises an elongated shaft attached to the balloon.

3. The method of claim 2, wherein the elongated shaft defines an inflation port in fluid communication with the balloon.

4. The method of claim 2, wherein the balloon defines a cylindrical shape.

5. The method of claim 2, wherein the balloon defines a rectangular shape.

6. The method of claim 2, wherein the balloon defines a triangular shape.

7. The method of claim 1, wherein the initial incision is formed with a distal tip of the stylet.

8. The method of claim 1, wherein the surface of the lateral wall comprises an interior surface defining a nasal cavity.

9. The method of claim 1, further comprising advancing a guide shaft adjacent to the surface of the lateral all of the nose.

10. The method of claim 9, wherein the guide shaft comprises a working channel, wherein the stylet is slidably housed within the working channel.

11. The method of claim 10, further comprising advancing an electrode assembly of the guide shaft against the incision and applying RF energy to the incision via the electrode assembly.

12. The method of claim 1, further comprising advancing the implant within the pocket of the lateral wall with a deployment mechanism.

13. The method of claim 12, wherein the deployment mechanism comprises a pair of jaws, wherein advancing the implant further comprises grasping the implant with the pair of jaws.

14. The method of claim 13, wherein placing the implant within the pocket of the lateral wall further comprises releasing the implant from the pair of jaws.

15. The method of claim 1, wherein the implant comprises a polymer.

16. The method of claim 1, wherein the implant comprises a plate forming a rectangular shape.

17. The method of claim 1, wherein the implant further comprises a therapeutic agent.

18. The method of claim 1, wherein the implant comprises a bioabsorbable material.

19. A method of inserting an implant within a lateral wall of a nose of a patient, the method comprising:
(a) forming a pocket within tissue in an interior portion of the lateral wall of the nose;
(b) expanding the pocket within the interior portion of the lateral wall of the nose with a balloon;
(c) inserting an implant within the expanded pocket.

20. A method of inserting an implant within a lateral wall of a nose of a patient, the method comprising:
(a) penetrating the lateral wall of the nose to thereby access an interior lining of the lateral wall of the nose;
(b) forming an expanded pocket within the interior lining of the lateral wall of the nose by inflating a balloon;
(c) deflating the balloon;
(d) removing the balloon from the pocket; and
(e) inserting an implant within the pocket after the pocket has been expanded by the balloon.
